(19) United States
(12) Patent Application Publication
(10) Pub. No.: US 2014/0187825 A1
(43) Pub. Date: Jul. 3, 2014

(54) LIGNOCELLULOSIC CONVERSION PROCESS INTENSIFICATION

(71) Applicants: Joseph B. Binder, Berkeley, CA (US); Jacob Borden, San Diego, CA (US); Michael Chappell, Encinitas, CA (US); Parag Shah, San Diego, IL (US)

(72) Inventors: Joseph B. Binder, Berkeley, CA (US); Jacob Borden, San Diego, CA (US); Michael Chappell, Encinitas, CA (US); Parag Shah, San Diego, IL (US)

(21) Appl. No.: 14/136,229

(22) Filed: Dec. 20, 2013

Related U.S. Application Data
(60) Provisional application No. 61/747,463, filed on Dec. 31, 2012.

(51) Int. Cl. C12P 19/14; C12P 7/14; C12P 19/02

(52) U.S. Cl. CPC C12P 19/14; C12P 19/02; C12P 7/14
USPC 568/840; 435/99; 435/162; 435/294.1

(57) ABSTRACT

Economic conversion of lignocellulose requires both the maximization of conversion of available carbohydrates, as well as minimization of process capital cost. Process intensification minimizes capital cost while preserving conversion yield by combining into a single step those unit operations that are conducted at similar conditions. Flowsheet variations are proposed that minimize process capital while maintaining overall conversion yield.

This application claims the benefit of U.S. Provisional Patent Application No. 61/747,463 filed on Dec. 31, 2012, which is hereby incorporated herein by reference in its entirety.

### BACKGROUND OF THE INVENTION

1. Technical Field

The invention relates to methods and systems related to renewable materials and biofuels production. Aspects of the invention relate to lignocellulosic conversion processes in which multiple process steps in a conventional process are combined in a single unit operation.

2. Discussion of Related Art

Lignocellulose processing involves multiple unit operations in order to economically convert available sugars, such as sucrose, hemicellulose, and cellulose, into desirable molecules, such as biofuels and biochemicals. For instance, a typical configuration for lignocellulose processing may involve the steps of feedstock preparation, hydrolysis, fermentation, and distillation. Collectively, these steps are usually taken in order to maximize the conversion yield of all available carbohydrates without formation of sometimes toxic side-products, such as furans or organic acids.

Biofuel production is desirable from an environmental standpoint, but in order to be commercialized, biofuel production must be economically feasible. High capital cost and overall economics of the process hinders commercialization. There is a need and desire to minimize capital in lignocellulose processing while maintaining conversion yields, particularly in the production of renewable materials such as biofuels.

### SUMMARY

The invention is directed to methods and systems for producing biofuels and other renewable materials, as well as biofuel component compositions made according to such methods. Compared to traditional processing of lignocellulosic biomass, the methods and systems described below result in minimized capital while maintaining or improving yields.

According to some embodiments, a method for production of renewable materials may include hydrolyzing a polysaccharide material while purifying the renewable material from a mixture that includes the polysaccharide material and the renewable material. For example, the polysaccharide may include hemicellulose and the hydrolyzing may include thermochemical hydrolysis. As another example, the renewable material may include a simple alcohol and the purifying may include distilling a portion of a fermentation broth. Furthermore, the method may include distillative hydrolysis occurring separate from fermenting processes using a lignocellulosic feedstock, wherein heat supplied drives distillation and hydrolysis.

The lignocellulosic feedstock used herein may include a hemicellulose material, a cellulose material, and a lignin material. Additionally, the lignocellulosic feedstock may include an unbound carbohydrate material. For example, the lignocellulosic feedstock may include sugarcane, energy cane, miscanthus, sorghum, sweet sorghum, Napier grass, corn stover, corn cobs, leaves, agricultural residue, switch grass, Arundo, energy grass, municipal solid waste, and/or hybrids thereof.

According to certain embodiments, a method of producing renewable materials from a lignocellulosic feedstock includes the steps of: (a) separating the renewable material from a fermentation broth while hydrolyzing a portion of the hemicellulose material to form a pentose material, (b) hydrolyzing at least a portion of the cellulose material to form a hexose material, and (c) fermenting the pentose material and the hexose material to produce fermentation broth comprising the renewable material.

In certain embodiments, the steps of hydrolyzing the cellulose material and fermenting the pentose material and the hexose material may occur substantially simultaneously. Also, in certain embodiments, the separating step may include distilling the renewable material.

According to additional embodiments, a method of producing renewable materials from a lignocellulosic feedstock includes: (a) fermenting the unbound carbohydrate material to produce renewable material in the presence of the hemicellulose material, the cellulose material, and the lignin material, (b) separating the renewable material from the hemicellulose material, the cellulose material, and the lignin material while hydrolyzing a portion of the hemicellulose material to form a pentose material, (c) hydrolyzing at least a portion of the cellulose material to form a hexose material, and (d) fermenting the pentose material and the hexose material to produce renewable material.

In accordance with the invention, there are a variety of ways in which the steps of this method may be carried out. In certain embodiments, step (c) and step (d) may occur substantially concurrently. Likewise, in certain embodiments, step (a) and step (d) may occur together. Furthermore, the renewable material of step (a) and step (d) may be combined before step (b). In certain embodiments, the lignin material may be separated between step (c) and step (d). Additionally or alternatively, heat supplied for separating the renewable material may also hydrolyze the hemicellulose material.

Renewable materials made by the methods herein may include a simple alcohol, for example. Examples of renewable materials made according to some embodiments include ethanol, n-butanol, isobutanol, 2-butanol, fatty alcohols, isobutene, isoprenoids, triglycerides, lipids, fatty acids, lactic acid, acetic acid, propanediol, and/or butanediol. The renewable material may include material suitable for use as biofuels, blendstocks, chemicals, intermediates, solvents, adhesives, polymers, and/or lubricants. The renewable material may include one or more biofuel components, such as lipids, or an alcohol, namely ethanol, butanol, and/or isobutanol. The biofuel may include gasoline, diesel, jet fuel, and/or kerosene.

According to some embodiments, a system for producing renewable materials from the lignocellulosic feedstock may include a distillative hydrolysis unit and a fermenting unit. The system may also include a lignin separation unit. In certain embodiments, the system may include a feedstock conditioning unit that includes a size reduction device. Additionally, in certain embodiments, the system may include a recycle line.

According to some embodiments, a method of producing renewable materials from a lignocellulosic feedstock may include: (a) fermenting in a first fermenting step the unbound carbohydrate material to produce renewable material in the presence of the hemicellulose material, the cellulose material, and the lignin material, (b) separating the renewable material from the hemicellulose material, the cellulose material, and the lignin material while hydrolyzing a portion of the hemicellulose material to form a pentose material, (c) fermenting in a second fermenting step a portion of the pentose material to produce renewable material in a fermentation broth with the lignin material, and (d) recycling a portion of the fermentation broth to the first fermenting step. In certain embodiments, a portion of the fermentation broth may be purged before step (d). The method may also include hydrolyzing the cellulose material to form a hexose material, and fermenting a portion of the hexose material to produce renewable material in a fermentation broth. Furthermore, in certain embodiments, the hydrolyzing and fermenting may occur substantially simultaneously with step (c).

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 is a process flow diagram illustrating a conventional method for producing a renewable material.

FIG. 2 is a process flow diagram illustrating one embodiment of a method for producing a renewable material using process intensification.

FIG. 3 is a process flow diagram illustrating another embodiment of a method for producing a renewable material using process intensification.

FIG. 4 is a process flow diagram illustrating yet another embodiment of a method for producing a renewable material using process intensification.

FIG. 5 is a process flow diagram illustrating still another embodiment of a method for producing a renewable material using process intensification.

### DETAILED DESCRIPTION

The invention is directed to methods and systems for producing biofuels and other renewable materials using a lignocellulosic conversion process that involves process intensification, as well as renewable materials made according to such methods.

As used herein, the term “renewable material” preferably refers to a substance and/or an item that has been at least partially derived from a source and/or a process capable of being replaced at least in part by natural ecological cycles and/or resources. Renewable materials may broadly include, for example, chemicals, chemical intermediates, solvents, adhesives, lubricants, monomers, oligomers, polymers, biofuels, biofuel intermediates, biogasoline, biogasoline blendstocks, biodiesel, green diesel, renewable diesel, biodiesel blend stocks, biodistillates, biochar, biocoke, renewable building materials, and/or the like. As a more specific example, the renewable material may include, without being limited to, any one or more of the following: ethanol, n-butanol, isobutanol, 2-butanol, fatty alcohols, isobutene, isoprenoids, triglycerides, lipids, fatty acids, lactic acid, acetic acid, propanediol, butanediol. In certain embodiments, the renewable material may include one or more biofuel components. For example, the renewable material may include a simple alcohol, such as ethanol, butanol, or isobutanol, or lipids.

The term “biofuel” preferably refers to components and/or streams suitable for use as a fuel and/or a combustion source derived at least in part from renewable sources. The biofuel can be sustainably produced and/or have reduced and/or no net carbon emissions to the atmosphere, such as when compared to fossil fuels. According to some embodiments, renewable sources can exclude materials mined or drilled, such as from the underground. In some embodiments, renewable resources can include single cell organisms, multi-cell organisms, plants, fungi, bacteria, algae, cultivated crops, non-cultivated crops, timber, and/or the like. Biofuels can be suitable for use as transportation fuels, such as for use in land vehicles, marine vehicles, aviation vehicles, and/or the like. More particularly, the biofuels may include gasoline, diesel, jet fuel, kerosene, and/or the like. Biofuels can be suitable for use in power generation, such as raising steam, exchanging energy with a suitable heat transfer media, generating syngas, generating hydrogen, making electricity, and/or the like.

“Lignocellulosic” and “lignocellulose” preferably broadly refer to materials containing cellulose, hemicellulose, lignin, and/or the like, such as may be derived from plant material and/or the like. Lignocellulosic material may include any suitable material, such as sugarcane, sugarcane bagasse, energy cane, energy cane bagasse, rice, rice straw, corn, corn stover, corn cobs, wheat, wheat straw, maize, maize stover, sorghum, sorghum stover, sweet sorghum, sweet sorghum stover, Arundo, cotton remnant, sugar beet, sugar beet pulp, soybean, rapeseed, jatropha, switch grass, energy grass, miscanthus, Napier grass, other grasses, and hybrids of any of these materials. Lignocellulosic material may also include, in general, grasses, leaves, legumes, forbs, cacti, timber, wood chips, softwoods such as pine and poplar, hardwoods such as eucalyptus, oak, and hickory, forest litter, wood waste, sawdust, paper, paper mill residue, paper waste, agricultural residue, municipal solid waste, any other suitable biomass material, and/or the like. Lignocellulosic feedstocks used in the methods and systems described herein typically include a hemicellulose material, a cellulose material, and a lignin material. In certain embodiments, the lignocellulosic feedstocks may also include an unbound carbohydrate material.

“Lignin” preferably broadly refers to a biopolymer that may be part of secondary cell walls in plants, such as a complex highly cross-linked aromatic polymer that may covalently link to hemicellulose.

“Hemicellulose” preferably broadly refers to a branched sugar polymer composed mostly of pentoses, such as with a generally random amorphous structure and typically may include up to hundreds of thousands of pentose units.

“Cellulose” preferably broadly refers to an organic compound with the formula (C₆H₁₀O₅)_{z} where z includes any suitable integer. Cellulose may include a polysaccharide with a linear chain of several hundred to over ten thousand hexose units and a high degree of crystalline structure, for example.

“Unbound carbohydrate” preferably broadly refers to sugar juice or sucrose that is not bound or not polymerized.

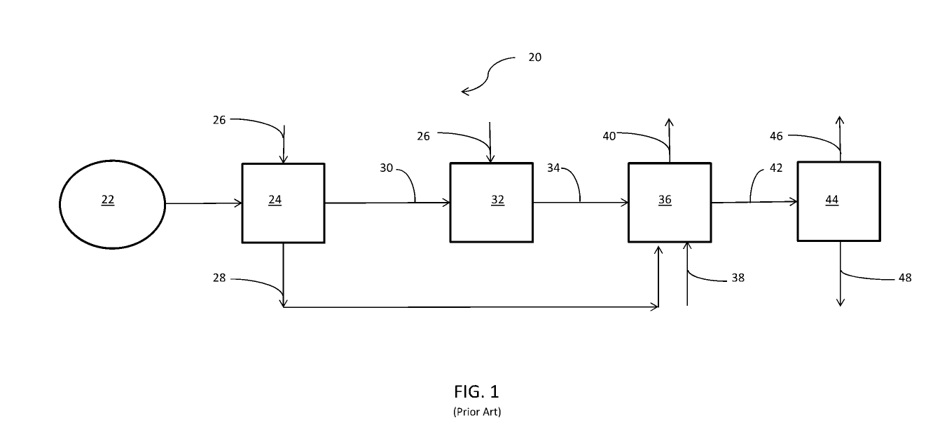

As explained above, lignocellulosic processing involves multiple unit operations in order to economically convert available sugars, such as sucrose, hemicellulose, and cellulose, into desirable molecules, such as biofuels and biochemicals. One example of a typical configuration for lignocellulose processing is illustrated by the flowsheet in FIG. 1. As shown in FIG. 1, the lignocellulose process 20 involves feeding a lignocellulosic feedstock 22 to a feedstock preparation unit 24 to which water 26 is also added. Sucrose juice 28 is extracted from the lignocellulosic feedstock 22 and water 26 in the feedstock preparation unit 24, and the sucrose juice 28 is fed to a fermentation unit 36 while the remaining fiber and water mixture 30 is fed to a hydrolyzer unit 32. More water 26 is fed to the hydrolyzer unit 32, which mixes with the fiber and water mixture 30. The hydrolyzed mixture 34 is then fed to the fermentation unit 36. In the fermentation unit 36, C6 and/or C5 enzymes 38 are added and CO₂ gas 40 is released. The fermented fiber mixture 42 is then fed to a distillation unit 44, from which the fermentation product 46, such as ethanol, may be obtained while the remaining fiber and water mixture 48 is removed.

These steps are usually taken in order to maximize the conversion yield of all available carbohydrates without formation of sometimes toxic side-products, such as furans or organic acids.

In order to minimize capital while maintaining conversion yields, the flowsheet illustrated in FIG. 1 can be optimized via process intensification. More particularly, consecutive or non-consecutive process steps can be combined in order to effect multiple outcomes in a single unit operation. Hence, capital minimization may be achieved by reducing the number of operations, thus eliminating the need for certain pieces of equipment.

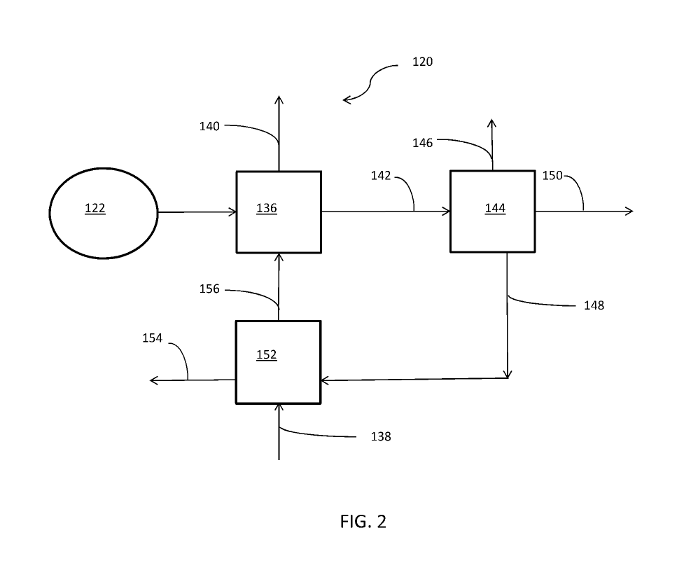

FIGS. 2-5, described in detail below, each embody a variation where distillation and hydrolysis are combined in order to effect “distillative hydrolysis,” which is the simultaneous recovery of fermentation product and pretreatment of lignocellulose for subsequent conversion. Further intensification could be realized with pre-conditioning of the lignocellulosic feedstock or by addition of base, such as NaOH or NH₄OH, or acid, such as H₂SO₄, to catalyze lignin and/or hemicellulose hydrolysis and solubilization. Addition of base or recycle of enzyme-containing solutions during distillative hydrolysis would help mitigate lignin re-precipitation with ethanol distillation.

The lignocellulose process illustrated by the flowsheet in FIG. 2 involves fermentation of soluble sugars, distillative hydrolysis, enzymatic saccharification with C6 and/or C5 enzymes, separation of non-digestible solids, and recycle of solubilized sugars to fermentation. More particularly, as shown in FIG. 2, the lignocellulose process 120 involves feeding a lignocellulosic feedstock 122 directly to a fermentation unit 136, along with recycled solubilized sugars 156, as explained below. As the soluble sugars are fermented, CO₂ gas 140 is released and the fermented fiber mixture 142 is then fed to a distillation unit 144 to undergo distillative hydrolysis. The fermentation product 146, which may be ethanol, is recovered while lignocellulose is pretreated for subsequent conversion. Water 150 is removed from the distillation unit 144 as the remaining fiber and water mixture 148 is passed along to an enzyme hydrolysis/separation unit 152. Additional water and C6 and/or C5 enzymes 138 are added to the enzyme hydrolysis/separation unit 152, in which enzymatic saccharification takes place, along with separation of non-digestible solids. The non-digestible solids 154 are removed from the process, while solubilized sugars 156 are recycled to the fermentation unit 136.

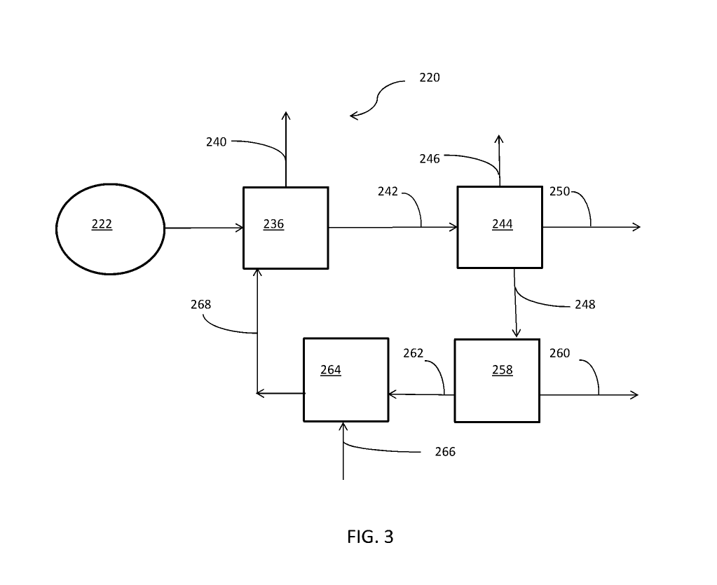

Like the lignocellulose process illustrated by the flowsheet in FIG. 2, the lignocellulose process illustrated by the flowsheet in FIG. 3 also involves fermentation of soluble sugars and distillative hydrolysis. The lignocellulose process illustrated by the flowsheet in FIG. 3 further includes separation of old/treated fibers and recycle of new/untreated fibers, enzymatic saccharification and/or simultaneous saccharification and fermentation (SSF) with C6 and/or C5 enzymes, and recycle of solubilized sugars and residual solids to fermentation. More particularly, as shown in FIG. 3, the lignocellulose process 220 involves feeding a lignocellulosic feedstock 222 directly to a fermentation unit 236 along with recycled solubilized sugars and residual solids 268, as explained below. As the soluble sugars are fermented, CO₂ gas 240 is released and the fermented fiber mixture 242 is then fed to a distillation unit 244 to undergo distillative hydrolysis. The fermentation product 246, which may be ethanol, is recovered while lignocellulose is pretreated for subsequent conversion. Water 250 is removed from the distillation unit 244 as the remaining fiber and water mixture 248, which includes both old/treated fibers and new/untreated fibers, is passed along to a solid/solid separation unit 258 that separates the old/treated fibers from the new/untreated fibers. The old/treated fibers 260 are removed from the process while the new/untreated fibers 262 are passed to an enzyme hydrolysis unit 264. C6 and/or C5 enzymes 266 are added to the enzyme hydrolysis unit 264, in which enzymatic saccharification and/or simultaneous saccharification and fermentation (SSF) with C6 and/or C5 enzymes takes place. The solubilized sugars and residual solids 268 are recycled to the fermentation unit 236.

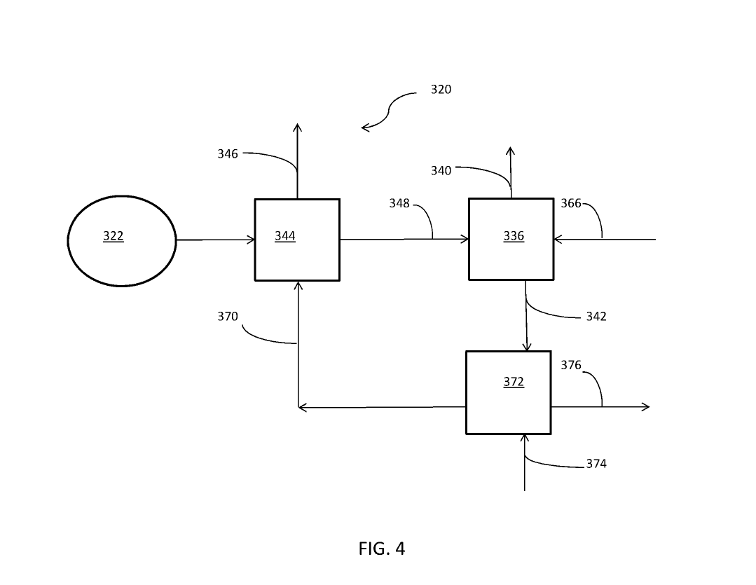

Unlike the lignocellulose processes illustrated by the flowsheets in FIG. 2 and FIG. 3, the lignocellulose process illustrated by the flowsheet in FIG. 4 involves distillative hydrolysis of the lignocellulosic feedstock, enzymatic saccharification and/or SSF with C6 and/or C5 enzymes with simultaneous fermentation of soluble sugars, separation of non-digestible solids, and recycle of recovered fermentation broth to distillative hydrolysis for product recovery. More particularly, as shown in FIG. 4, the lignocellulose process 320 involves feeding a lignocellulosic feedstock 322 directly to a distillation unit 344, along with recovered fermentation broth 370, as explained below, to undergo distillative hydrolysis. The fermentation product 346, which may be ethanol, is recovered while lignocellulose is pretreated for subsequent conversion. The remaining fiber and water mixture 348 is passed along to a fermentation unit 336. C6 and/or C5 enzymes 366 are added to the fermentation unit 336, as enzymatic saccharification and/or SSF with C6 and/or C5 enzymes occurs simultaneously with fermentation of the soluble sugars. As the soluble sugars are fermented, CO₂ gas 340 is released and the fermented fiber mixture 342 is then fed to a solid/liquid separation unit 372 to which water 374 is also added. Following separation, the recovered fermentation broth 370 is recycled to the distillation unit 344, while the solids 376 are removed from the process.

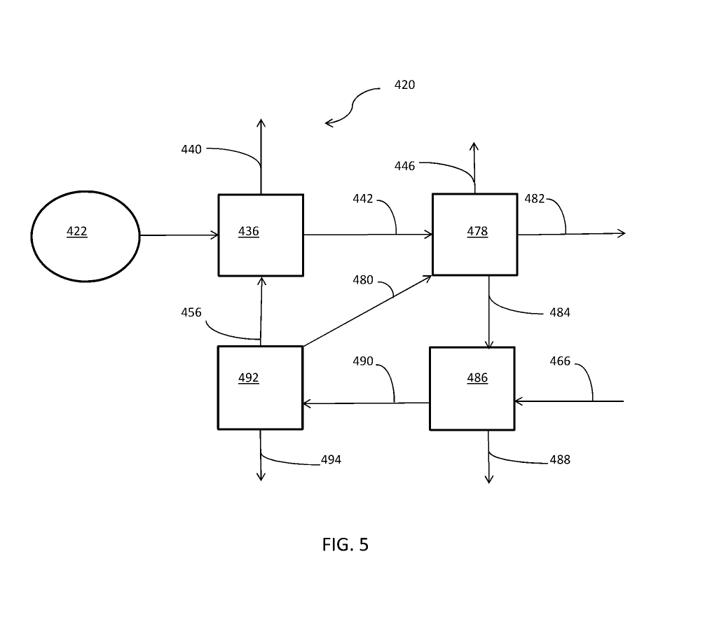

The lignocellulose process illustrated by the flowsheet in FIG. 5 uses distillative hydrolysis with two beer columns, which involves fermentation of soluble sugars, enzymatic saccharification of C6 and/or C5 enzymes and fermentation, product recovery and solid/liquid separation, and recycle of water and unfermented unbound carbohydrates and solid fibers. As shown in FIG. 5, the lignocellulose process 420 involves feeding a lignocellulosic feedstock 422 directly to a first degree fermentation unit 436 for sucrose conversion along with recycled solubilized sugars 456, as explained below. As the soluble sugars are fermented, CO₂ gas 440 is released and the fermented fiber mixture 442 is then fed to a product recovery and hydrolysis unit 478 to undergo distillative hydrolysis. Recycled water and unfermented unbound carbohydrates 480, described below, are also fed to the product recovery and hydrolysis unit 478. The fermentation product 446, which may be ethanol, is recovered while lignocellulose is pretreated for subsequent conversion. Water 482 is removed from the product recovery and hydrolysis unit 478 as the remaining fiber and water mixture 484 is passed along to an enzyme hydrolysis and second degree fermentation unit 486. C6 and/or C5 enzymes 466 are added to the enzyme hydrolysis and second degree fermentation unit 486, in which both enzymatic saccharification of C6 and/or C5 enzymes and fermentation takes place. As the soluble sugars are fermented, CO₂ gas 488 is released and the hydrolyzed and fermented fiber mixture 490 is then fed to a product recovery and liquid/solid separation unit 492. The solubilized sugars 456 are recycled to the fermentation unit 436, while the water and unfermented unbound carbohydrates 480 are recycled to the product recovery and hydrolysis unit 478, and the non-digestible solids 494 are removed from the process.

In any of the lignocellulose processes described herein, the solids removed from the process may be put to use, such as in power generation, recycled products, or waste treatment. For example, the solids may either be burned to generate steam and electricity, which may be sold to the grid to improve GHG balance, or they used for quality particle board, fiber products, or waxes, for example.

Additional flowsheet variants for process intensification may be used in combination with any of the illustrated flowsheets, such as “evaporative fermentation,” wherein vacuum separation of fermentation off-gases effects carbohydrate conversion simultaneously with product recovery.

In a more general embodiment, a method for producing renewable materials includes hydrolyzing a polysaccharide material while purifying the renewable material from a mixture that includes both the polysaccharide material and the renewable material. For example, the polysaccharide may include hemicellulose and the hydrolyzing may include thermochemical hydrolysis. Additionally, the renewable material may include a simple alcohol and the purifying may be carried out by distilling a portion of a fermentation broth. In this type of embodiment, the method may include distillative hydrolysis that occurs separate from fermenting processes using a lignocellulosic feedstock, wherein heat supplied drives distillation and hydrolysis.

As a more specific example, a method of producing renewable materials from a lignocellulosic feedstock may include separating the renewable material from a fermentation broth while hydrolyzing a portion of the hemicellulose material to form a pentose material; hydrolyzing at least a portion of the cellulose material to form a hexose material; and fermenting the pentose material and the hexose material to produce fermentation broth comprising the renewable material. Optionally, hydrolyzing the cellulose material and fermenting the pentose material and the hexose material may occur substantially simultaneously. Furthermore, separating the renewable material from the fermentation broth may include distilling the renewable material.

As another example, a method of producing renewable materials from a lignocellulosic feedstock may include: fermenting an unbound carbohydrate material to produce renewable material in the presence of hemicellulose material, cellulose material, and lignin material; separating the renewable material from the hemicellulose material, the cellulose material, and the lignin material while hydrolzying a portion of the hemicellulose material to form a pentose material; hydrolyzing at least a portion of the cellulose material to form a hexose material; and fermenting the pentose material and the hexose material to produce renewable material.

In certain embodiments, the steps of hydrolyzing the cellulose material and fermenting the pentose material and the hexose material may occur substantially concurrently.

In certain embodiments, the steps of fermenting the unbound carbohydrate material and fermenting the pentose material and the hexose material occur together.

In certain embodiments, the renewable material from the step of fermenting the unbound carbohydrate material and the renewable material from the step of fermenting the pentose material and the hexose material may be combined before carrying out the step of separating the renewable material from the hemicellulose material, the cellulose material, and the lignin material while hydrolzying a portion of the hemicellulose material to form a pentose material.

Certain embodiments may include separating the lignin material between the steps of hydrolyzing the cellulose material and fermenting the pentose material and the hexose material.

In any of the described methods, heat supplied for separating the renewable material may also hydrolyze the hemicellulose material.

In general, a system used herein for producing renewable materials from lignocellulosic feedstock may include a distillative hydrolysis unit and a fermenting unit, as illustrated in FIGS. 2-5. The system may also include a lignin separation unit. As another option, the system may include a feedstock conditioning unit that includes a size reduction device. Furthermore, the system may include a recycle line.

As yet another example of a method of producing renewable materials from a lignocellulosic feedstock, the method may include: fermenting in a first fermenting step an unbound carbohydrate material to produce renewable material in the presence of hemicellulose material, cellulose material, and lignin material; separating the renewable material from the hemicellulose material, the cellulose material, and the lignin material while hydrolyzing a portion of the hemicellulose material to form a pentose material; fermenting in a second fermenting step a portion of the pentose material to produce renewable material in a fermentation broth with the lignin material; and recycling a portion of the fermentation broth to the first fermenting step.

In certain embodiments, a portion of the fermentation broth may be purged before the step of recycling a portion of the fermentation broth to the first fermenting step.

Certain embodiments may also include hydrolyzing at least a portion of the cellulose material to form a hexose material; and fermenting a portion of the hexose material to produce renewable material in a fermentation broth. These steps may occur substantially simultaneously with the step of fermenting in a second fermenting step a portion of the pentose material to produce renewable material in a fermentation broth with the lignin material.

According to some embodiments, the invention may be directed to a renewable material made according to any of the methods and/or systems described herein.

While distillative hydrolysis, for example, achieves capital minimization by enabling the recovery of ethanol vapors in the same equipment where fiber pretreatment occurs, thus reducing the overall number of unit operations (and in some embodiments, eliminating roller mills, hydrolysis, and/or detox sections), distillative hydrolysis and the other process intensification arrangements described herein also provide a number of secondary benefits. These secondary benefits include:
Better alignment with C5 de-polymerization/degradation kinetics; enablement of two-stage pretreatment; reduced inhibitor/aldehyde formation
Improved operability by maintaining solid:liquid ratio>3-3.5 throughout the plant
Bio-burden control by lowering incoming feed and fermenting at pH of about 4-4.5, and by conducting separate hydrolysis and fermentation (SHF) at 70-80° C.
Alignment with C5/C6 enzyme temperature optimum for improved SHF kinetics
Reduction in pH and temperature oscillations across the plant; lower acid/base use; lower osmotic/salt stress
Elimination of acetate inhibition by acid-catalyzed esterification to ethyl acetate co-product; recovery in distillative hydrolysis section

The following Example compares ethanol yield of five different methods based on the flowsheets illustrated in FIGS. 1-5.

### Example

As described above, FIG. 1 is a process flow diagram illustrating a conventional method for producing a renewable material. In this example, the data in Table 1, below, corresponds to each of the stages of the flow diagram in FIG. 1. The overall yield in this example is 86.00 gallons/dry MT.

TABLE 1
Data Corresponding to FIG. 1

| | |
|---|---|
| Lignocellulosic feedstock (22) | 1 kg feedstock: |
| | 17 wt % digestible fibers |
| | 10 wt % non-digestible fibers |
| | 3 wt % sucrose |
| | 70 wt % water |
| | 2.33 liquid:solid ratio |
| Feedstock preparation unit (24) | 1 Imbib rate |
| Water (26) added to feedstock | 0.168 kg water |
| preparation unit | |
| Sucrose juice (28) | 0.63 kg sucrose juice: |
| | 5 wt % sucrose |
| | 95 wt % water |
| Fiber and water mixture (30) | 0.53 kg |
| | 31 wt % digestible fibers |
| | 19 wt % non-digestible fibers |
| | 0 wt % sucrose |
| | 50 wt % water |
| | 1.00 liquid:solid ratio |
| Hydrolyzer unit (32) | 2 liquid:solid ratio |
| Water (26) added to hydrolyzer unit | 0.27 kg water |
| Hydrolyzed mixture (34) | 0.80 kg |
| | 21 wt % digestible fibers |
| | 12 wt % non-digestible fibers |
| | 0 wt % sucrose |
| | 67 wt % water |
| | 2.00 liquid:solid ratio |
| Fermentation unit (36) | 45 wt % ethanol yield |
| | 82.3% digestible fiber conversion |
| CO₂ gas (40) | 0.09 kg CO₂ |
| Fermented fiber mixture (42) | 1.34 kg |
| | 9.6 wt % residual fiber |
| | 5.7 wt % ethanol |
| | 84.6 wt % water |
| | 9.41 liquid:solid ratio |
| Fermentation product (46) | 0.08 kg ethanol |
| Remaining fiber and water | 1.26 kg |
| mixture (48) | 10.2 wt % residual fiber |
| | 89.8 wt % water |
| | 8.81 liquid:solid ratio |

As described above, FIG. 2 is a process flow diagram illustrating one embodiment of a method for producing a renewable material using process intensification. In this example, the data in Table 2, below, corresponds to each of the stages of the flow diagram in FIG. 2. The overall yield in this example is 86.85 gallons/dry MT.

TABLE 2
Data Corresponding to FIG. 2

| | |
|---|---|
| Lignocellulosic feedstock (122) | 1 kg feedstock: |
| | 17 wt % digestible fibers |
| | 10 wt % non-digestible fibers |
| | 3 wt % sucrose |
| | 70 wt % water |
| | 2.3 liquid:solid ratio |
| | 35° Celsius |
| | 3.98 kJ/kg-K |
| Fermentation unit (136) | 45 wt % fermentation yield |
| CO₂ gas (140) | 0.094 kg CO₂ offgas |
| Fermented fiber mixture (142) | 2.09 kg |
| | 8.1 wt % digestible fiber |
| | 4.7 wt % non-digestible fiber |
| | 3.7 wt % ethanol |
| | 83.4 wt % water |
| | 4.3% beer titer |
| | 5.0 liquid:solid ratio |
| | 35° Celsius |
| | 4.07 kJ/kg-K |
| Fermentation product (146) | 0.08 kg ethanol |
| | 100 wt % ethanol |
| Water (150) removed from distillation | 0.67 kg water |
| unit | 100 wt % water |
| | 4.18 kJ/kg-K |
| Remaining fiber and water mixture (148) | 1.34 kg |
| | 12.6 wt % digestible fibers |
| | 7.4 wt % non-digestible fibers |
| | 80.0 wt % water |
| | 4.0 liquid:solid ratio |
| | 160° Celsius |
| | 4.04 kJ/kg-K |
| Enzyme hydrolysis/separation unit (152) | 82.0% digestible fiber |
| | conversion |
| Water and C6 and/or C5 enzymes (138) | 0.10 kg imbib water |
| Non-digestible solids (154) | 0.26 kg bleed |
| | 11.9 wt % digestible fibers |
| | 38.6 wt % non-digestible |
| | fibers |
| | 50.0 wt % water |
| | 1.00 liquid: solid ratio |
| | 160° Celsius |
| | 3.86 kJ/kg-K |
| Solubilized sugars (156) | 1.18 kg |
| | 0 wt % digestible fibers |
| | 0 wt % non-digestible fibers |
| | 12 wt % sugar |
| | 88 wt % water |

As described above, FIG. 3 is a process flow diagram illustrating another embodiment of a method for producing a renewable material using process intensification. In this example, the data in Table 3, below, corresponds to each of the stages of the flow diagram in FIG. 3. The overall yield in this example is 86.5 gallons/dry MT.

TABLE 3
Data Corresponding to FIG. 3

| | |
|---|---|
| Lignocellulosic feedstock (222) | 1 kg feedstock: |
| | 17 wt % digestible fibers |
| | 10 wt % non-digestible fibers |
| | 3 wt % sucrose |
| | 70 wt % water |
| Fermentation unit (236) | 45 wt % fermentation yield |
| CO₂ gas (240) | 0.094 kg CO₂ offgas |
| Fermented fiber mixture (242) | 2.24 kg |
| | 0.27 kg new fiber |
| | 63 wt % digestible new fiber |
| | 37 wt % non-digestible new fiber |
| | 0.13 kg old fiber |
| | 23 wt % digestible old fiber |
| | 78 wt % non-digestible old fiber |
| | 0.08 kg ethanol |
| | 4.2% beer titer |
| | 1.76 kg water |
| | 78.9 wt % moisture |
| | 4.47 liquid:solid ratio |
| Fermentation product (246) | 0.08 kg ethanol |
| | 100 wt % ethanol |
| Water (250) removed from | 0.18 kg water |
| distillation unit | 100 wt % water |
| Remaining fiber and water | 1.97 kg |
| mixture (248) | 0.27 kg new fiber |
| | 62.9 wt % digestible new fiber |
| | 37.0 wt % non-digestible new fiber |
| | 0.13 kg old fiber |
| | 22.8 wt % digestible old fiber |
| | 77.5 wt % non-digestible old fiber |
| | 1.58 kg water |
| | 80 wt % moisture |
| | 4.00 liquid:solid ratio |
| Solid/solid separation unit (258) | 99% old reject; new retain |
| Old/treated fibers (260) | 0.64 kg bleed |
| | 0.00 kg new fiber |
| | 63 wt % digestible new fiber |
| | 37 wt % non-digestible new fiber |
| | 0.13 kg old fiber |
| | 22.8 wt % digestible old fiber |
| | 77.5 wt % non-digestible old fiber |
| | 0.52 kg water |
| | 4.00 liquid:solid ratio |
| New/untreated fibers (262) | 1.33 kg |
| | 0.26 kg new fiber |
| | 63 wt % digestible new fiber |
| | 37 wt % non-digestible new fiber |
| | 0.00 kg old fiber |
| | 23 wt % digestible old fiber |
| | 78 wt % non-digestible old fiber |
| | 1.06 kg water |
| | 80 wt % moisture |
| | 4.00 liquid:solid ratio |
| Enzyme hydrolysis unit (264) | 82.3 wt % digestible fiber conversion |
| Solubilized sugars and residual | 1.33 kg |
| solids (268) | 0.13 kg new fiber |
| | 23 wt % digestible new fiber |
| | 77 wt % non-digestible new fiber |
| | 0.0010 kg old fiber |
| | 5.0 wt % digestible old fiber |
| | 95.0 wt % non-digestible old fiber |
| | 0.14 kg sugar |
| | 1.06 kg water |
| | 80 wt % moisture |
| | 0.80 liquid:solid ratio |

As described above, FIG. 4 is a process flow diagram illustrating yet another embodiment of a method for producing a renewable material using process intensification. In this example, the data in Table 4, below, corresponds to each of the stages of the flow diagram in FIG. 4. The overall yield in this example is 86.4 gallons/dry MT.

TABLE 4
Data Corresponding to FIG. 4

| | |
|---|---|
| Lignocellulosic feedstock (322) | 1 kg feedstock: |
| | 17 wt % digestible fibers |
| | 10 wt % non-digestible fibers |
| | 3 wt % sucrose |
| | 70 wt % water |
| | 2.3 liquid:solid ratio |
| Fermentation product (346) | 0.77 kg ethanol |
| Remaining fiber and water mixture | 1.67 kg |
| (348) | 10.1 wt % digestible fiber |
| | 5.9 wt % non-digestible fiber |
| | 2.0 wt % sucrose |
| | 0.0 wt % ethanol |
| | 82.0 wt % water |
| | 0.0% beer titer |
| | 5.1 liquid:solid ratio |
| | 35° Celsius |
| | 4.06 kJ/kg-K |
| Fermentation unit (336) | 82% digestible fiber conversion |
| | 45% fermentation yield |
| CO₂ gas (340) | 0.095 kg CO₂ offgas |
| Fermented fiber mixture (342) | 1.58 kg |
| | 1.9 wt % digestible fiber |
| | 6.3 wt % non-digestible fiber |
| | 4.9 wt % ethanol |
| | 0.0 wt % sucrose |
| | 86.9 wt % water |
| | 5.3% beer titer |
| | 6.7 liquid:solid ratio |
| | 35° Celsius |
| | 4.09 kJ/kg-K |
| Solid/liquid separation unit (372) | 2 liquid:solid ratio |
| Water (374) added to solid/liquid | 0.258 kg water |
| separation unit | |
| Recovered fermentation broth (370) | 0.75 kg |
| | 0.0 wt % digestible fiber |
| | 0.0 wt % non-digestible fiber |
| | 10.3 wt % ethanol |
| | 0.0 wt % sucrose |
| | 89.7 wt % water |
| | 10.3% beer titer |
| | N/A liquid:solid ratio |
| | 35° Celsius |
| | 4.11 kJ/kg-K |
| Removed solids (376) | 1.09 kg |
| | 2.8 wt % digestible fiber |
| | 9.1 wt % non-digestible fiber |
| | 0.0 wt % ethanol |
| | 0.0 wt % sucrose |
| | 88.1 wt % water |
| | 0.0% beer titer |
| | 7.4 liquid:solid ratio |
| | 35° Celsius |
| | 4.10 kJ/kg-K |

As described above, FIG. 5 is a process flow diagram illustrating still another embodiment of a method for producing a renewable material using process intensification. In this example, the data in Table 5, below, corresponds to each of the stages of the flow diagram in FIG. 5. The overall yield in this example is 90 gallons/dry MT.

TABLE 5
Data Corresponding to FIG. 5

| | |
|---|---|
| Lignocellulosic feedstock (422) | 1 kg feedstock: |
| | 17 wt % digestible fibers |
| | 10 wt % non-digestible fibers |
| | 3 wt % sucrose |
| | 70 wt % water |
| | 2.3 liquid:solid ratio |
| | 35° Celsius |
| | 3.98 kJ/kg-K |
| First degree fermentation unit (436) | 45 wt % fermentation yield |
| CO₂ gas (440) | 0.02 kg CO₂ offgas |
| Fermented fiber mixture (442) | 1.98 kg |
| | 8.7 wt % digestible fiber |
| | 6.7 wt % non-digestible fiber |
| | 1 wt % ethanol |
| | 80 wt % water |
| | 7 liquid:solid ratio |
| | 35° Celsius |
| | 4.04 kJ/kg-K |
| Fermentation product (446) | 0.09 kg ethanol |
| | 100% ethanol |
| Water (482) removed from product | 0.19 kg water |
| recovery and hydrolysis unit | 100% water |
| Remaining fiber and water mixture | 1.91 kg |
| (484) | 5.2 wt % digestible fiber |
| | 6.2 wt % non-digestible fiber |
| | 1.2 wt % unbound carbohydrate |
| | 80 wt % water |
| | 11 liquid:solid ratio |
| | 180° Celsius |
| | 4.04 kJ/kg-K |
| CO₂ gas (488) | 0.07 kg CO₂ offgas |
| Hydrolyzed and fermented fiber mixture | 1.84 kg |
| (490) | 1 wt % digestible fiber |
| | 6.2 wt % non-digestible fiber |
| | 4 wt % ethanol |
| | 82.6 wt % water |
| | 22 liquid:solid ratio |
| | 35° Celsius |
| | 4.06 kJ/kg-K |
| Solubilized sugars (456) | 1 kg |
| | 0.4 wt % digestible fiber |
| | 5 wt % non-digestible fiber |
| | 1.6 wt % unbound carbohydrate |
| | 89 wt % water |
| | 37° Celsius |
| | 4.1 kJ/kg-K |
| Recycled water and unfermented | 0.22 kg |
| unbound carbohydrates (480) | 30 wt % ethanol |
| | 70 wt % water |
| Removed non-digestible solids (494) | 0.62 kg |
| | 3 wt % digestible fiber |
| | 20 wt % non-digestible fiber |
| | 77 wt % water |
| | 6.8 liquid:solid ratio |

The method illustrated by the flowsheet in FIG. 5 has the highest product yield compared to the other configurations. The primary difference between this configuration and other configurations is the absence of a solid/liquid separation step immediately after hydrolysis or fermentation, which prevents the loss of unbound carbohydrates or ethanol.

It will be apparent to those skilled in the art that various modifications and variations can be made in the disclosed structures and methods without departing from the scope or spirit of the invention. Particularly, descriptions of any one embodiment can be freely combined with descriptions or other embodiments to result in combinations and/or variations of two or more elements or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. A method for production of renewable materials, the method comprising hydrolyzing a polysaccharide material while purifying the renewable material from a mixture comprising the polysaccharide material and the renewable material.

2. The method of claim 1, wherein the polysaccharide comprises hemicellulose and the hydrolyzing comprises thermochemical hydrolysis.

3. The method of claim 1, wherein the renewable material comprises a simple alcohol and the purifying comprises distilling a portion of a fermentation broth.

4. Method of claim 1, wherein the method comprises distillative hydrolysis occurring separate from fermenting processes using a lignocellulosic feedstock, wherein heat supplied drives distillation and hydrolysis.

5. Renewable material made by the method of claim 1.

6. A method of producing renewable materials from a lignocellulosic feedstock, wherein the lignocellulosic feedstock comprises a hemicellulose material, a cellulose material, and a lignin material, the method comprising:
a) separating the renewable material from a fermentation broth while hydrolyzing a portion of the hemicellulose material to form a pentose material;
b) hydrolyzing the cellulose material to form a hexose material; and
c) fermenting the pentose material and the hexose material to produce fermentation broth comprising the renewable material.

7. The method of claim 5, wherein step b) and step c) occur substantially simultaneously.

8. The method of claim 5, wherein the separating comprises distilling the renewable material.

9. A method of producing renewable materials from a lignocellulosic feedstock, wherein the lignocellulosic feedstock comprises an unbound carbohydrate material, a hemicellulose material, a cellulose material, and a lignin material, the method comprising:
a) fermenting the unbound carbohydrate material to produce renewable material in the presence of the hemicellulose material, the cellulose material, and the lignin material;
b) separating the renewable material from the hemicellulose material, the cellulose material, and the lignin material while hydrolzying a portion of the hemicellulose material to form a pentose material;
c) hydrolyzing the cellulose material to form a hexose material;
d) fermenting the pentose material and the hexose material to produce renewable material.

10. The method of claim 9, wherein step c) and step d) occur substantially concurrently.

11. The method of claim 9, wherein step a) and step d) occur together.

12. The method of claim 9, further comprising combining the renewable material of step a) and step d) before step b).

13. The method of claim 9, further comprising separating the lignin material between step c) and step d).

14. The method of claim 9, wherein heat supplied for separating the renewable material also hydrolyses the hemicellulose material.

15. The method of claim 9, wherein the lignocellulosic feedstock comprises sugarcane, energy cane, miscanthus, sorghum, sweet sorghum, Napier grass, corn stover, corn cobs, leaves, agricultural residue, switch grass, Arundo, energy grass, or municipal solid waste.

16. Renewable material made by the method of claim 15.

17. The composition of matter of claim 16, wherein the renewable material comprises a simple alcohol.

18. A system for producing renewable materials from the lignocellulosic feedstock, the system comprising:
a distillative hydrolysis unit; and
a fermenting unit.

19. The system of claim 18, further comprising a lignin separation unit.

20. The system of claim 18, further comprising a feedstock conditioning unit comprising a size reduction devise.

21. The system of claim 18, further comprising a recycle line.
